(19) United States
(12) Patent Application Publication
(10) Pub. No.: US 2007/0191702 A1
(43) Pub. Date: Aug. 16, 2007

(54) Systems and methods for sensing analyte and dispensing therapeutic fluid

(75) Inventors: Ofer Yodfat, Maccabim-Reut (IL); Ruthy Kaidar, Hafia (IL)

Correspondence Address: MINTZ LEVIN COHN FERRIS GLOVSKY & POPEO, 666 THIRD AVENUE, NEW YORK, NY, 10017, US

(73) Assignee: MEDINGO LTD., Yoqneam Illit (IL)

(21) Appl. No.: 11/706,606

(22) Filed: Feb. 14, 2007

Related U.S. Application Data
(60) Provisional application No. 60/773,842, filed on Feb. 15, 2006.

(51) Int. Cl. A61N 1/30; A61B 5/00; A61B 5/05

(52) U.S. Cl. 600/365; 600/316; 600/347; 604/19

(57) ABSTRACT

Systems and methods are provided for sensing analyte (e.g., glucose) and/or dispensing therapeutic fluid (e.g., insulin). The systems and methods are based on transporting the therapeutic fluid through a cannula at least a portion of which is permeable to molecule of the analyte. Sensing and detection of the concentration level of the analyte can be carried out by optical sensing, electrochemical sensing, acoustical sensing etc. Sensing and dispensing can be carried out by sensing and dispensing device operating in either closed or semi-closed loop.

### CROSS REFERENCE TO RELATED APPLICATION

This application claims the benefit of U.S. provisional patent application No. 60/773,842, filed Feb. 15, 2006, which is hereby incorporated by reference herein in its entirety.

### FIELD OF THE INVENTION

Embodiments of the present invention relate generally to methods and devices for regulation of glucose levels. More particularly, some embodiments of the invention concern a system comprising a glucose sensor, an insulin dispenser and a processor-controller, which assesses the sensed glucose levels and programs the dispenser for delivering an adjustable amount of insulin to the human body (i.e., a closed loop system). Even more particularly, some embodiments of the present invention relate to miniature, single piece, portable devices, that can be directly attached to a patient's skin (for example), which may include one exit port, designed for concomitantly sensing glucose and dispensing insulin. Embodiments of the present invention employ available methods for accurately sensing glucose levels and for controlling dispensing of insulin. It should be borne in mind that the present invention is not limited strictly for delivering insulin and sensing glucose. Within the scope of the present invention are a method and a system for delivering of any other drug and for concomitantly sensing an analyte, which is not necessarily glucose. When used in the following description the term “analyte” means any solute composed of specific molecules dissolved in an aqueous medium.

### BACKGROUND OF THE INVENTION

Diabetes and Glycemic Control

Diabetes mellitus is a disease of major global importance, increasing in frequency at almost epidemic rates, such that the worldwide prevalence is predicted to at least double to about 300 million people over the next 10-15 years. Diabetes is characterized by a chronically raised blood glucose concentration (hyperglycemia), due to a relative or absolute lack of the pancreatic hormone, insulin. The normal pancreatic islet cells (beta cells) continuously sense the blood glucose levels and consequently regulate insulin secretion to maintain near constant levels.

Much of the burden of the disease to the patient and to health care resources is due to the long-term tissue complications, which affect both the small blood vessels (microangiopathy, causing eye, kidney and nerve damage) and the large blood vessels (causing accelerated atherosclerosis, with increased rates of coronary heart disease, peripheral vascular disease and stroke). There is now evidence that morbidity and mortality of diabetic patients is related to the duration and severity of hyperglycemia (DCCT Trial, N Engl J Med 1993; 329: 977-986, UKPDS Trial, Lancet 1998; 352: 837-853. BMJ 1998; 317, (7160): 703-13 and the EDIC Trial, N Engl J Med 2005; 353, (25): 2643-53). In theory, returning blood glucose levels to normal by replacement insulin injections and other treatments in diabetes should prevent complications, but, frustratingly, near-normal blood glucose concentrations are very difficult to achieve and maintain in many patients, particularly those with type 1 diabetes. In these patients, blood glucose levels can swing between high and low (hypoglycemia) in an unpredictable manner. Thus, in order to achieve tight glycemic control, the two functions of the normal pancreas, glucose sensing and insulin delivery, both should be substituted. A closed loop system provided with a feedback mechanism could theoretically maintain near normal blood glucose levels.

Insulin Delivery

Recently, intensive therapies that include multiple daily injections (MDI) or insulin pump therapy have been prescribed with the goal of maintaining nearly normal blood glucose levels to avoid long term complications.

Multiple daily injections: MDI insulin regimens require three or more daily injections. These injections are typically made up of a combination of long-acting insulin with multiple doses of rapid acting insulin.

Pump therapy: Pump therapy is one of the most technologically advanced methods of achieving near normal blood glucose levels, and there are at least four reasons in favor of using the pump to intensify treatment. First, insulin is absorbed in a more stable manner which may lead to improved glycemic control over MDI (Diabetes Care 2003; 16: 1079-1087, Diabetes Care 2005; 28: 533-538). Second, studies have shown a decreased risk of the “dawn phenomenon,” which is a common rise in blood glucose before breakfast, and better control throughout the night (Diabetes Care 2002; 25: 593-598). Third, the insulin pump gives patients more flexibility in the timing of their meals. Patients on the pump can adjust for snacks and meals, as well as for exercise and physical exertion. Finally, studies have shown that the pump reduces the occurrence of serious hypoglycemic episodes (Pediatrics 2001; 107: 351-356).

These devices represent a significant improvement over multiple daily injections, but they suffer from several drawbacks. One such drawback is the device's large size and weight, due to the spatial configuration of the syringe and piston together with a relatively large driving mechanism. The relatively bulky device should be carried in the patient's pocket or attached to the patient's belt.

Consequently the fluid delivery tube is long (usually >40 cm) to allow insertion at remote sites. The uncomfortable bulky device with a long tube was rejected by the majority of diabetic insulin users because it disturbs daily activities (sleeping, swimming, physical activities and sex) and has unacceptable effect on teenagers' body image. In addition, the delivery tube excludes additional optional remote insertion sites like buttocks and extremities. Examples of first generation disposable syringe type reservoir fitted with tubes were described in 1972 by Hobbs in U.S. Pat. No. 2,631,847, in 1973 by Kaminski in U.S. Pat. No. 3,771,694 and later by Julius in U.S. Pat. No. 4,657,486 and by Skakoon in U.S. Pat. No. 4,544,369. To avoid the tubing limitations, a new concept (second generation) was proposed and described in prior art. The pump in accordance with this concept comprises a housing having a bottom surface adapted to be in contact with the skin of the patient, a reservoir disposed within the housing and an injection needle adapted to connect with the reservoir. This paradigm was described by Schneider in U.S. Pat. No. 4,498,843, Burton in U.S. Pat. No. 5,957,895, Connelly in U.S. Pat. No. 6,589,229 and Flaherty in U.S. Pat. Nos. 6,740,059 and 6,749,587.

Glucose Monitoring

Most diabetic patients currently measure their own blood glucose several times during the day by obtaining finger-prick capillary samples and applying the blood to a reagent strip for analysis in a portable meter. Whilst blood glucose self-monitoring has had a major impact on improving diabetes care in the last few decades, the disadvantages of this technology include the discomfort of obtaining a blood sample leading to non-compliance.

Testing cannot be performed during sleeping or when the subject is occupied (e.g. during driving a motor vehicle), and intermittent testing may miss episodes of hyper- and hypoglycemia. The ideal glucose monitoring technology should therefore employ automatic and continuous testing.

Currently in-vivo continuous monitoring can be done by semi invasive means. The sensors are implanted in the subcutaneous tissue and measure interstitial fluid (ISF) glucose concentrations, which correspond blood glucose levels in the steady state (Diabetologia 1992; 35, (12): 1177-1180) but lag behind when glycemia is changing rapidly, for example after a meal. The magnitude of this lag time has been variously recorded in numerous studies with needle-type enzyme electrodes in animal and human studies over the last 20 years and found to range from about 5 to 30 min (Diabetologia 1986; 29: 817-821, Acta Diabetol 1993; 30: 143-148 and Am J Physiol. 2000; 278: E716-E728).

Currently there are three commercially available in vivo continuous glucose sensors, which make use of different technologies: 1—Glucose oxidase based sensors are described in U.S. Pat. No. 6,360,888 (Collin) and U.S. Pat. No. 6,892,085 (McIvor) assigned to Medtronic MiniMed Inc. (CGMS, Guardian™ and CGMS Gold), and U.S. Pat. No. 6,881,551 (Heller) assigned to Abbott Laboratories, formerly TheraSense, Inc., (Navigator™). These sensors consist of a subcutaneously implanted, needle-type amperometric enzyme electrode, coupled with a portable logger. The data can be downloaded from the logger to a portable computer after up to 3 days of sensing (Diab Technol Ther 2000; 2: (Suppl. 1), 13-18). The sensor is based on the long-established technology of glucose oxidase immobilized at a positively charged base electrode, with electrochemical detection of hydrogen peroxide produced. Aside from lag, there exist at least two other problems with subcutaneously implanted enzyme electrodes. These problems are unpredictable drift and impaired responses in vivo, which necessitate repeated calibration against finger-prick capillary blood glucose concentrations about four times daily. The accuracy of this technique using the Clarke error grid is apparently good, with about 95% of non-calibration paired blood and sensor values falling in the clinically acceptable zones A or B (Biosensors and Bioelectronics 2005; 20, (10): 1897-1902).

2—Reverse iontophoresis based sensors as detailed in U.S. Pat. No. 6,391,643 (Chen) assigned to Cygnus, Inc. (GlucoWatch™). A small current passed between two skin-surface electrodes draws ions (by electro-endosmosis) and glucose-containing interstitial fluid to the surface and into hydrogel pads incorporating a glucose oxidase biosensor (JAMA 1999; 282: 1839-1844). Readings in the latest version are taken every 10 min, with a single capillary blood calibration. The disadvantages of these sensors are occasional times when sensor values differ markedly from blood values as well as skin rash and skin irritation under the device in many patients, a long warm up time of 3 h and skips due to sweating.

3—The third commercial technology in current clinical use is based on microdialysis (Diab Care 2002; 25: 347-352) as detailed in U.S. Pat. No. 6,091,976 (Pfeiffer) assigned to Roche Diagnostics. There exists also a commercial device (Menarini Diagnostics, GlucoDay™). Here, a fine, hollow dialysis fiber is implanted in the subcutaneous tissue and perfused with isotonic fluid. Glucose from the tissue diffuses into the fiber and is pumped outside the body for measurement by a glucose oxidase-based electrochemical sensor. Initial reports (Diab Care 2002; 25: 347-352) show good agreement between sensor and blood glucose readings, and good stability with a one-point calibration over one day. In fact better accuracies have been achieved by the microdialysis method as compared to the methods employing subcutaneous glucose oxidase sensor (Diabetes Care 2005; 28, (12): 2871-6).

Closed Loop Systems

In an artificial pancreas, sometimes referred to as a “closed loop” system, the continuous glucose sensor would report the blood glucose value to the insulin pump, which would then calculate and deliver the appropriate dosage of insulin. Since the advent of the insulin pump in the late 1970s, there has been a way to deliver insulin continuously. In sharp contrast to diabetes therapy today, the person with diabetes would in no way be involved with decision-making. An artificial pancreas is also expected to have the power to eliminate debilitating episodes of hypoglycemia, particularly nighttime hypoglycemia. In fact, even a simple turn-off feature in which a rapidly dropping or low blood glucose value halts the delivery of insulin to prevent hypoglycemia. An intermediate step in the way to achieve a “closed loop” system is an “open loop” (or “semi-closed loop”) system also called “closed loop with meal announcement”. In this model, user intervention is required, as the person with diabetes “boluses” in a way similar to today's insulin pumps, by keying in the desired insulin before they eat a meal. This would minimize the time lag problem (due to delays in ISF sensing and subcutaneous absorption time), but it would not have some of the advantages of a closed loop, as there would still be user involvement. “Open loop” systems have successfully been used in hospital settings with improved morbidity and mortality rates (ROSSO Trial, Diabetologia 2005: Dec. 17: 1-8) and in intensive care units (the CLINICIP approach). However these systems are not portable and are in use for bedridden patients only.

Communication between portable blood glucometers (requiring ex-vivo blood measurement) and insulin pumps are described in U.S. Pat. No. 5,338,157 (Blomquist). In these systems each glucose measurement is downloaded manually (usually remotely) by the patient to the pump for data storage only. The introduction of external continuous glucose monitoring systems described above allows for the first time continuous transmission of ISF glucose levels (sensing arm) to the insulin pump (dispensing arm) attaining a closed loop system. An example of a portable closed loop system is described in U.S. Pat. No. 6,558,351 (Steil) assigned to Medtronic MiniMed Inc.

In these systems the sensor and pump are two discrete components with separate housing, where both relatively bulky and heavy devices should be attached to the patient's belt. In addition, the two devices require two infusion sets with long tubing, two insertion sites, consequently extending the system's insertion and disconnections time and substantially increasing adverse events like infections, irritations, bleeding, etc.

In view of the foregoing, there is a need for improved systems and methods for sensing analyte and dispensing therapeutic fluid.

### SUMMARY OF THE INVENTION

Embodiments of the present invention relate to systems and methods for sensing analyte and/or dispensing fluid to the body of a mammal. Some embodiments of the present invention relate to devices that include both a sensing apparatus and a dispensing apparatus. The dispensing apparatus may be used for infusing fluid into the mammal's body, which may be a medication administered to a patient. The sensing apparatus may be used for detection of analytes via one or more measurements of analyte concentration. The dispensing apparatus and the sensing apparatus may be used together in a closed loop system, in which a processor-controller apparatus regulates the dispensing of fluid according to the sensed analyte concentration. In some embodiments, the dispensed fluid may be insulin that is administered to a diabetic patient and the analyte may be glucose.

In an illustrative embodiment, an external and optionally at least partially disposable apparatus is provided that functions as an artificial pancreas. The apparatus may be miniature, hidden under the clothes, and directly attachable to a patient's skin, avoiding tubing and allowing normal daily life activities (including swimming, shower, sports, etc.) without necessitating periodical disconnections.

In some embodiments, an apparatus is provided for in vivo detection of an analyte (e.g., glucose). The apparatus may include at least one housing (e.g., a cutaneously adherable patch), at least one cannula, a sensor, and a pump (e.g., peristaltic pump). The cannula may include a proximal portion located within the housing and a distal portion located external to the housing, where the distal portion is configured for subcutaneous placement within a mammal's body and at least a portion of the cannula is permeable to molecules of an analyte. The sensor may be configured to detect a concentration level of the analyte within the cannula. For example, the sensor may be located at least partially within the housing and may be configured to detect a concentration level of the analyte within the proximal portion of the cannula. The sensor may detect a concentration level of the analyte at about, or subsequent to the establishing of a concentration equilibrium between the analyte within the cannula and the analyte outside the cannula. In some embodiments, memory may be provided within the housing for storing measurements from the sensor continuously or at predetermined intervals. The pump may reside in the housing and may be adapted to transport a fluid (e.g., a therapeutic fluid such as insulin, a non-therapeutic fluid such as saline, or a combination thereof) to the mammal's body.

In some embodiments, osmotic pressure may be the driving force for urging glucose molecules to move across the cannula semi-permeable membrane. Alternatively or additionally, a mechanism (e.g., peristaltic pump) may be provided for drawing the analyte to a space within the cannula.

In some embodiments, the housing may additionally include a processor and a reservoir for the fluid. The pump may be in fluid communication with the reservoir and in electrical communication with the processor, and the pump may be configured to dispense a perfusate fluid to the mammal's body in an amount based at least in part on a signal received from the processor.

In some embodiments, the cannula may include an opening (e.g., at its distal end) and the pump may be configured to dispense the therapeutic fluid to the mammal's body through the opening.

In other embodiments, the apparatus may include a second cannula, and the pump may be configured to dispense the therapeutic fluid to the mammal's body through the second cannula.

In some embodiments, the sensor may include at least one of an optical sensor, an electrochemical sensor, and an acoustic sensor. For optical sensing, the sensor may detect concentration level of the analyte based on an optical detection method selected from the group of optical detection methods consisting of near infra red (“NIR”) reflectance, mid infra red (“IR”) spectroscopy, light scattering, Raman scattering, fluourescence measurements, and a combination thereof.

In some embodiments, the distal portion of the cannula may be configured for subcutaneous placement within a location of the mammal's body that provides access to interstitial fluid (“ISF”). In some embodiments, the distal portion of the cannula may be configured for subcutaneous placement within a location of the mammal's body that provides access to blood. For example, the cannula may be embedded within bodily tissue including blood vessels, a peritoneal cavity, muscle and the like.

In some embodiments, the sensor and the pump may operate in a closed-loop configuration. In other embodiments, the sensor and the pump operate within a semi-closed loop configuration upon external input. For example, a user may provide external input into the system regarding meal intake with the respective amount of the fluid needed to be administered to the user's body. The processor-controller may then use both the input from the sensing device and from the user to compute the amount of fluid to be pumped out of the dispensing system and into the patient's body.

In some embodiments, methods are provided for in vivo detection of an analyte. A cannula may be provided, wherein at least a portion of the cannula is permeable to molecules of an analyte (e.g., glucose). The cannula may be positioned at least partially subcutaneously within a mammal. A concentration level of the analyte may be sensed within the cannula at about, or subsequent to establishing an equilibrium between a concentration level of the analyte within the cannula and a concentration level of the analyte outside the cannula. A fluid (e.g., insulin) may be transported to the mammal's body (e.g., based at least in part on the sensed concentration level of the analyte). In some embodiments, the transporting of the fluid may be carried out through the same cannula that is used for the sensing of the analyte concentration. In other embodiments, a second cannula may be provided through which the fluid is transported to the mammal's body.

### BRIEF DESCRIPTION OF THE DRAWINGS

For a better understanding of the present invention, reference is made to the following description, taken in conjunction with the accompanying drawings, in which like reference numerals refer to like parts throughout, and in which:

FIG. 1 is a schematic drawing of a closed loop system, including a dispensing apparatus, a sensing apparatus and a processor-controller apparatus, with a single exit port;

FIG. 2 is a schematic drawing of a closed loop system, including a dispensing apparatus, a sensing apparatus and a processor-controller apparatus with multiple exit ports;

FIG. 3 illustrates an example of an embodiment according to the present invention;

FIGS. 4A and 4B illustrate an example of a penetrating member, cannula and well assembly;

FIG. 5A illustrates an example of the penetrating member and cannula of FIGS. 4A and 4B after insertion into the body, through the well assembly, according to one embodiment of the present invention;

FIG. 5B illustrates the embodiment of FIGS. 4A and 4B after removal of the penetrating member;

FIG. 6 illustrates sensing apparatus subassemblies according to embodiments of the present invention;

FIG. 7 illustrates sensing apparatus subassemblies, with a well assembly, according to embodiments of the present invention;

FIG. 8 illustrates a detailed view of a cannula according to an embodiment of the present invention;

FIG. 9 illustrates a cannula with an analyte-rich dialysate, and a sensing device, according to an embodiment of the invention;

FIG. 10 illustrates an example of a sensing apparatus using an optical sensing device according to an embodiment of the present invention;

FIG. 11 illustrates an example of a fully semi-permeable cannula according to an embodiment of the present invention;

FIG. 12 illustrates an example of a cannula comprising two separate materials, connected mechanically, according to an embodiment of the present invention; and

FIG. 13 is a drawing of a measurement cell and a glucose sensor according to an embodiment of the present invention in which electrochemical glucose oxidase based sensing is performed.

### DETAILED DESCRIPTION OF THE INVENTION

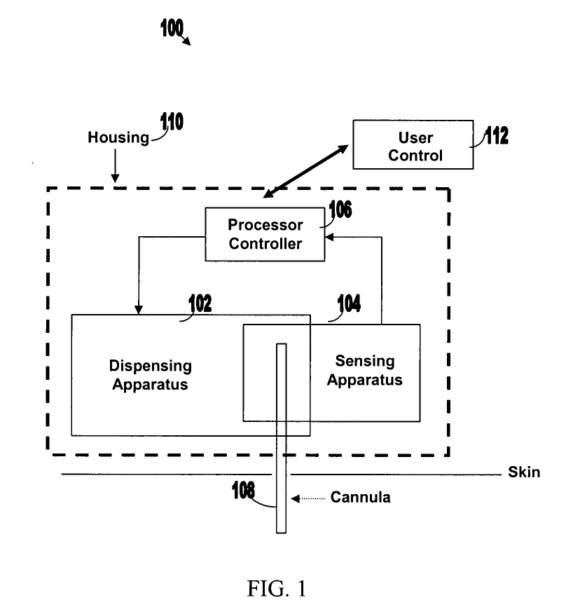

FIG. 1 illustrates various components of an exemplary closed loop system 100. Closed loop system 100 within the dashed frame may include dispensing apparatus 102, sensing apparatus 104, processor-controller apparatus 106 and cannula 108. All units are preferably enclosed within a single, common housing 110, which can be attached to the patient's skin. A single cannula 108, comprising a tubular body including a semi-permeable membrane, may be used to penetrate the skin and allows both fluid delivery to the patient's body and sensing of analytes within the patient's body. Processor-controller apparatus 106 can receive inputs from the sensing apparatus (i.e. analyte concentration) and after processing the data, may control the dispensing apparatus to dispense fluid accordingly.

The semi-closed loop (open loop) may include, in addition to the components disclosed for the closed-loop system, user control unit 112 (shown outside housing 110). This unit may be used for remote or direct programming and/or data handling of the processor-controller apparatus. Furthermore this unit allows visual display of the data or informing the user by the available means. For example, processor-controller apparatus 106 (which may include one or more processors) may receive inputs from the sensing apparatus and from the user control unit allowing simultaneous data processing of the user and sensor inputs and control of the dispensing of fluid accordingly.

In one embodiment, the dispensed fluid is insulin, the analyte is glucose and the body compartment is the subcutaneous interstitial fluid (ISF). In the closed loop system, insulin may be continuously (or in short intervals, usually every 3-10 minutes) dispensed to the subcutaneous compartment through the cannula. Insulin may reside in the cannula during the short interval while it is being delivered to the patient's body and during inter-delivery intervals. The cannula allows penetration of ISF glucose across its semi-permeable membrane into the insulin residing within it, achieving equilibrium in glucose concentrations. The sensing apparatus may measure the glucose concentration within the upper part of the cannula (which is proportional to the ISF glucose concentration).

The dispensed insulin emerging from the cannula in short intervals continuously washes the cannula to avoid cannula occlusion. Processor-controller apparatus 106 can receive the measured ISF glucose levels from the sensor and using a specified criteria (e.g., software code that takes into consideration lag periods due to slow absorption rates), controls the dispensing apparatus to adjust insulin dispensing according to ISF glucose levels. In the semi-closed loop system, processor-controller 106 may receive the measured glucose level from the sensor in addition to inputs from the patient (either changes in basal insulin delivery rates or boluses before meals) and accordingly controls the dispensing apparatus to deliver required insulin quantities to maintain normal glucose levels.

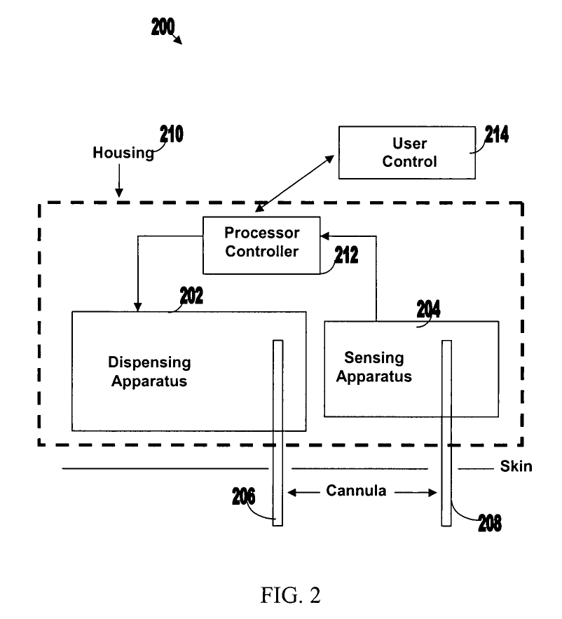

FIG. 2 illustrates another embodiment of a system 200 in accordance with the present invention. In this embodiment, dispensing apparatus 202 and sensing apparatus 204 have separate cannulae (206 and 208, respectively), thus two cannulae emerge from the same housing 210. Dispensing apparatus 202 may include one or more features of an insulin pump as described in prior art (e.g., reservoir, driving mechanism, tubing, etc.) and cannula 206, which is preferably not permeable. Sensing apparatus 204 may comprise a reservoir containing fluid and a pump for dispensing the fluid through the semi-permeable cannula, allowing glucose level measurements as described above. Processor-controller unit 212 may receive inputs from the sensing apparatus and from the patient (via user control unit 214 in the semi-closed loop configuration) and accordingly may control the dispensing apparatus to deliver insulin through the respective cannula to regulate glucose levels. The control unit 214 may also display the results of glucose level measurements.

In another embodiment, the dispensing apparatus and/or the sensing apparatus may be placed away from the patient's skin and held in the patient's pocket, belt, or any other desirable location, at the patient's convenience. In these configurations there may be separate housings for the dispensing apparatus and the sensing apparatus. The processor-controller apparatus may reside in both parts and input/output data can be delivered wirelessly or by any physical communication means.

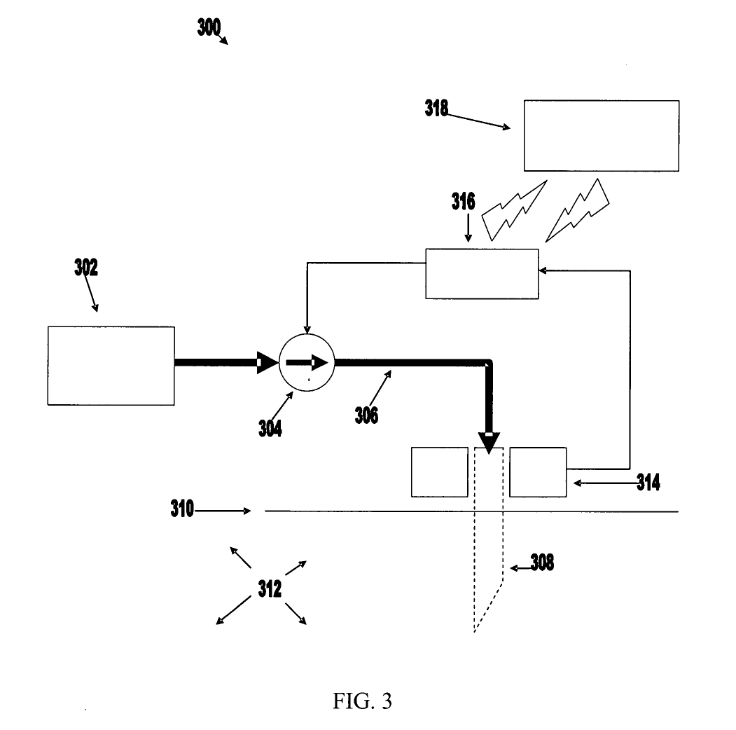

FIG. 3 shows one example of an embodiment of a system 300 in accordance with the present invention. As illustrated, the dispensing apparatus may comprise a reservoir 302 which contains a fluid to be dispensed (e.g., insulin), pump 304 which dispenses the fluid from reservoir, tube 306 through which the fluid passes from the pump, and semi-permeable cannula 308 penetrating the user's skin 310, allowing fluid delivery into the user's body 312, e.g. into the subcutaneous tissue. The sensing apparatus may comprise a sensing device 314 that measures the desired constituent concentration (e.g. glucose) within the upper portion of the cannula 308. The semi-permeable cannula 308 preferably allows free movement of molecules below a predetermined size (i.e. smaller than glucose) to achieve concentration equilibrium between the concentration measured in the body compartment and the concentration of the fluid within the cannula. The concentration of the molecules can be measured in the upper portion of the cannula by the sensing device 314. The processor-controller apparatus 316 may receive inputs from the sensing apparatus, process the data and control the dispensing apparatus to deliver fluid according to a predetermined algorithm, thus forming a closed loop system.

In another embodiment of the system, user control unit 302 containing a user interface (button, display, etc.) enables programming and data collection, either directly or wirelessly. In this embodiment, processor-controller 316 can operate according to commands generated by an outside source, e.g. the control unit 318, allowing a user to give operation commands to the processor-controller and thus to determine the flow rate profile manually. As in the previous embodiments the control unit 318 allows visual display of the data or informing the user by the available means.

In another embodiment, processor-controller 316 can receive inputs from the sensing apparatus in addition to “on demand” inputs from the patient by the user control unit 318, thus allowing a semi-closed loop (open loop) system.

As known to one of ordinary skill in the art, the dispensing apparatus can comprise various types of reservoirs (e.g. syringe type, bladder, cartridge), various pumping mechanisms (e.g. peristaltic pump, plunger movement within a syringe, etc.) and various driving mechanisms (e.g. DC or stepper motors, SMA derived motors, piezo, bellow, etc.). In addition, the cannula can be inserted by a penetrating member (which is removed after skin pricking) and brought in fluid communication with a conducting tube 306 through a well assembly, for example, as described in our Israel patent application number IL171813.

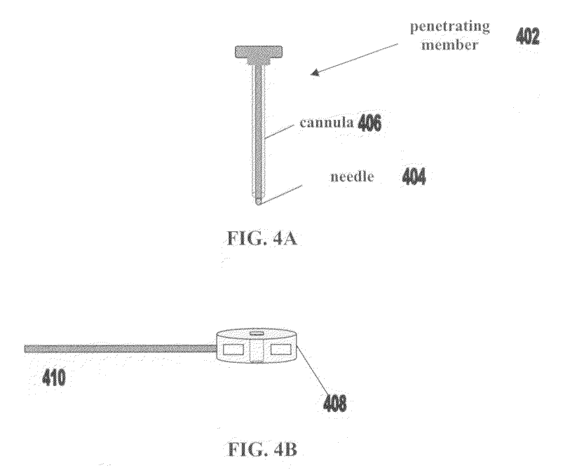

FIG. 4A illustrates an example of an assembly that includes penetrating member 402 (with needle 404) and cannula 406 in accordance with an embodiment of the present invention. FIG. 4B illustrates an example of a well assembly. The well assembly may include the well itself 408 and tubing 410 leading fluid to the well.

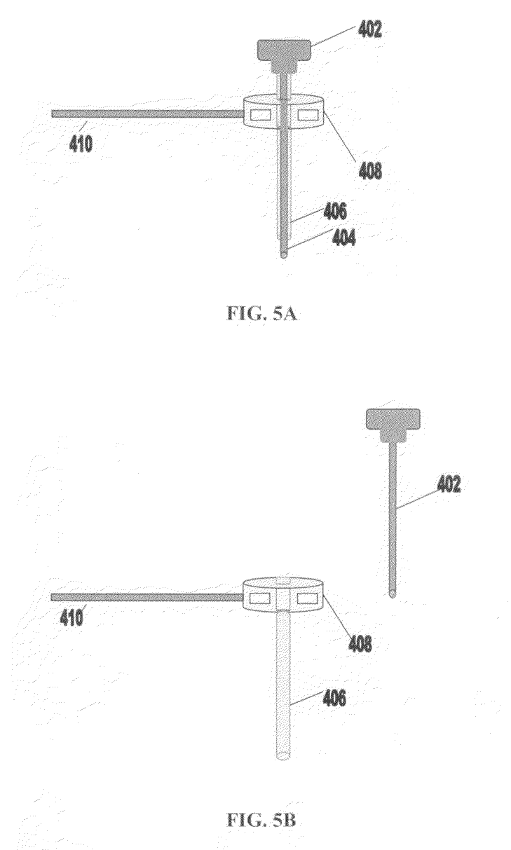

FIG. 5A illustrates an example of the penetrating member 402 and cannula 406 after insertion into the body, through the well assembly 408 before removal of penetrating member 402. FIG. 5B illustrates the system after removal of the penetrating member 402. Cannula 406 may be insertable subcutaneously within the body in a usual matter after puncturing the skin by a penetrating member. Cannula 406 may comprise a tubular body fitted with a lateral inlet port and with an outlet port. The fluid, e.g. insulin, may be supplied to the cannula through the lateral port and may be delivered to the body through the outlet port. The cannula body may be at least partially made of semi-permeable material, to allow for diffusion or microdialysis of molecules of an analyte, e.g. glucose, from the body into the cannula.

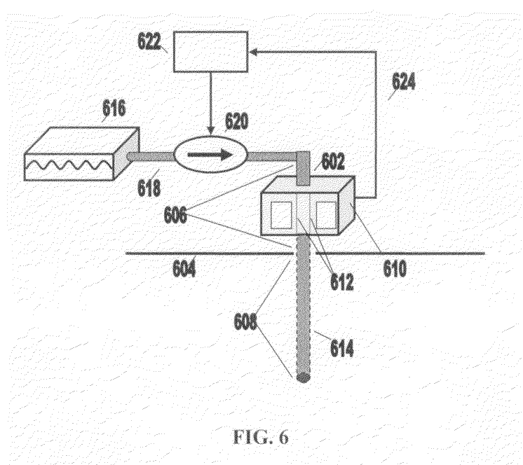

FIG. 6 illustrates examples of sensing apparatus subassemblies according to some embodiments for the present invention. The fluid may be delivered from the dispensing apparatus via the cannula 602, which punctures the skin 604, into the user's body. The cannula may comprise two portions—an upper cannula portion 606, residing above the skin 604, and a lower cannula portion 608, residing below the skin 604, with the opening of the cannula residing within the body tissue. The sensing device 610 may be used to measure analyte concentration within the fluid residing in a portion (e.g., a designated portion) of the cannula, serving as measurement cell 612. The walls of the lower portion of the cannula can be made of a semi-permeable membrane 614. This membrane is preferable for establishing an analyte concentration equilibrium between both sides of the membrane. FIG. 6 also shows a reservoir 616, tube 618, pump 620, and processor-controller 622, as previously described. Sensing device 610 may send feedback signals to processor-controller 622 via path 624.

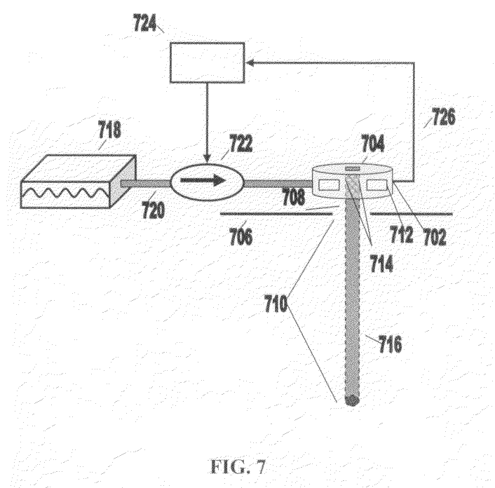

FIG. 7 illustrates examples of sensing apparatus subassemblies, with a well assembly. The fluid may be delivered from the dispensing apparatus to well assembly 702, which serves as a small basin of fluid through which the cannula 704 passes before puncturing the skin 706 and delivering fluid into the user's body. The cannula may comprise two portions—an upper cannula portion 708, residing above the skin 706, and a lower cannula portion 710, residing below the skin 706, with the opening of the cannula residing within the body tissue. The sensing device 712 may reside within the well assembly 702 and may be used to measure analyte concentration within the fluid residing in a portion 714 of the cannula, referred to as a measurement cell. The walls of the lower part of the cannula can be made of a semi-permeable membrane 716 to allow for the establishment of an analyte concentration equilibrium between both sides of the membrane. FIG. 7 also shows a reservoir 718, tube 720, pump 722, and processor-controller 724, as previously described. Sensing device 712 may send feedback signals to processor-controller 724 via path 726.

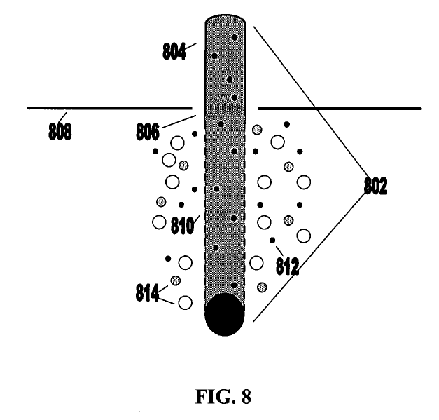

FIG. 8 illustrates schematically an embodiment of a semi-permeable cannula 802, with its upper 804 and lower 806 portions residing correspondingly above and below the skin 808, and a schematic view of the diffusion, or dialysis process. At least the lower cannula portion 806 may comprise a semi-permeable membrane 810 to allow substances of low molecular weight, and particularly, the desired analyte(s) (e.g., glucose) 812 to pass through pores of the semi-permeable membrane 810, while higher molecular weight substances 814 do not pass through. The cannula 802 may be perfused with a fluid (also called the perfusate) like insulin or saline. Diffusion of analyte molecules occurs across the semi-permeable membrane 810, due to, for example, the initial concentration gradient. To that end, the diffusion, or dialysis, process occurs in the direction of the concentration gradient, from the tissue (e.g. ISF) into the solution within the cannula finally reaching equilibrium in analyte concentrations between the inner and outer sides of the cannula. The solution enriched by the analyte is called the dialysate. The outcome of this diffusion, or dialysis, process is the presence of a dialysate inside the cannula 802 with an analyte concentration which is proportional to the analyte concentration in the tissue.

In one embodiment, the suitable membrane 810 is a semi-permeable membrane which could be used for microdialysis. The suitable membrane may be defined by the following properties: pores that allow the molecule of interest to pass, a constant, well-defined area available for diffusion, or dialysis, and biocompatibility.

The cutoff level of a dialysis membrane (e.g., the size of pores and/or other parameters), determines what kind of substances (with regard to molecular weight) will pass through pores of the membrane and be accumulated in the dialysate. Thus, substances with molecular weights surpassing the cutoff level remain in the interstitial space and are excluded from entering the dialysate.

In one embodiment of the present invention, a microdialysis cannula is provided which is a microdialysis probe that also serves as a cannula, and which may not necessarily be removed after insertion into the body.

Microdialysis probes are well-known in the art and examples may be found in U.S. Pat. No. 4,694,832 (Ungerstedt), as well as from the CMA/Microdialysis AB company, under the name “CMA 60 Microdialysis Catheter” or “CMA 70 Brain Microdialysis Catheters”. A microdialysis probe coupled with a cannula for insertion is also described in published U.S. application no. 20050119588 A1. The present embodiment of a microdialysis cannula may be similar to the above mentioned microdialysis probe, apart from the fact that it is preferably open at the bottom. Thus, the cannula in this embodiment, serves both as a means for dispensing fluid into the body and as a microdialysis probe for measuring analyte concentrations.

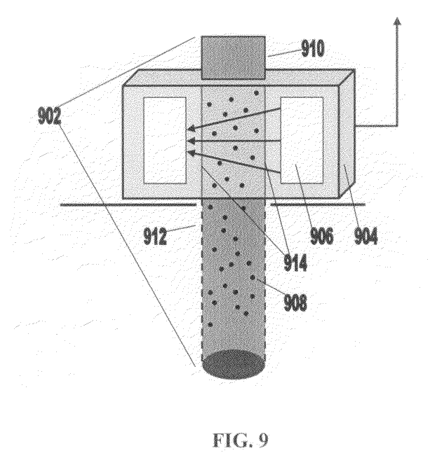

FIG. 9 illustrates an embodiment of a cannula 902 with the analyte-rich dialysate, and a sensing device 904 including one or more sensors 906. In the present embodiment, the analyte, the low molecular weight substance, may allow to pass through the semi-permeable membrane is glucose 908 and the solution used as perfusate in the microdialysis, or diffusion, process is insulin. The sensing device can be used as a stand alone item, when it is required only to sense the level of an analyte. For the sake of brevity, the reservoir with the fluid perfusing the cannula and the pumping means are omitted. After the diffusion process takes place and equilibrium is established, the dialysate, enriched with the analyte (e.g. glucose) 908, resides inside the entire cannula 902, where both the upper 910 and lower 912 portions of the cannula 902 contain the dialysate. Particularly, the upper cannula portion 910, which resides above the skin, serves as a measurement cell 914. Transportation of the analyte towards the measurement cell 914 can be enhanced by a suitable means such as, for example, by a peristaltic pump. This measurement cell 914 confines the location where the analyte concentration measurement takes place. The concentration is measured according to the analyte levels in the dialysate.

In one embodiment, the measurement cell 914 is made of a transparent or translucent material facilitating utilization of optical detection methods in the sensing device 904, for analyte (e.g., glucose) level measurements. The measurement cell may reside in the upper cannula portion 910 above the body and preferably does not come in contact with any internal biological tissues that may occlude the transparency of the measurement cell and affect its optical properties.

In another embodiment, the fluid, which serves as a perfusate in the microdialysis (diffusion) process, is insulin and the analyte is glucose. This facilitates the application of optical methods for the detection of glucose concentration. However, one should bear in mind, that in accordance with some embodiments of the present invention other drugs can be used for perfusing the cannula instead of insulin and other analytes can be sensed instead or in addition to glucose.

In embodiments in which the measurement cell is transparent and an optical method is used for detection of glucose concentration levels, the sensing apparatus may use an optical sensor 904 which surrounds the measurement cell. The optical sensor operates according to optical detection methods, using a means of illumination applied to the dialysate residing in the measurement cell, and a means of detection for determining analyte concentration. An example of such an embodiment may include a measurement cell which serves as an analyte-filled cuvette. Analyte concentration can be determined for example by known in the art spectrophotometric methods.

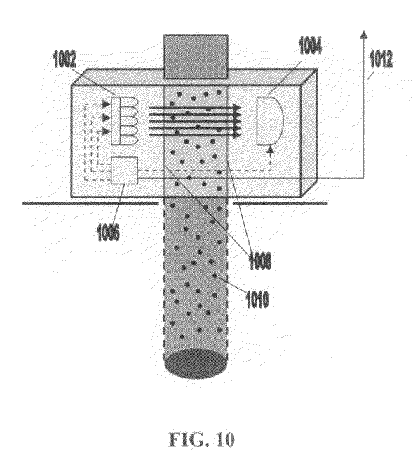

FIG. 10 illustrates an example of a sensing apparatus using an optical sensor comprising a set of light emitting diodes (LEDs) 1002 as a means for illumination and an Indium Gallium Arsenide (InGaAs) sensor (1004) as a means for detection. Provided also may be a processing means 1006 (e.g., one or more processors), which controls functioning of the LEDs and of the detector. The analyte resides in the measurement cell 1008 which is positioned between the LEDs and the InGaAs sensor. The optical sensor detects the concentration level of the analyte (e.g., glucose) 1010 in the dialysate and sends an appropriate feedback signal 1012 to the processor-controller apparatus.

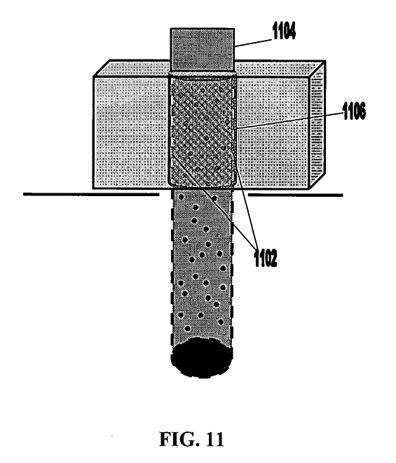

In one preferred embodiment, the entire cannula, including the lower and upper cannula portions, may include a semi-permeable membrane. FIG. 11 shows an example of such a fully semi-permeable cannula. In one embodiment of the measurement cell 1102, the upper cannula portion 1104 is embraced by a transparent or translucent casing 1106. This casing leaves the upper portion transparent and at the same time prevents the leakage of dialysate from the cannula.

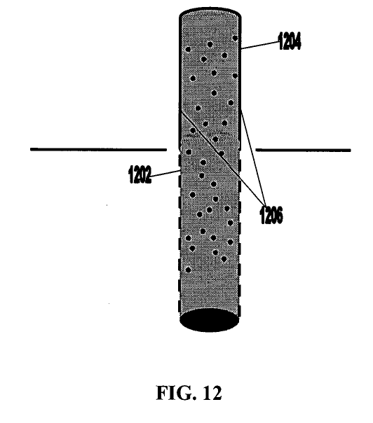

FIG. 12 shows another embodiment in which the cannula comprises two separate portions—a lower cannula portion 1202, which comprises a semi-permeable membrane and an upper cannula portion 1204, which is not permeable and is made of a transparent or translucent material, suitable for connection to the lower cannula portion. The portions can be attached by gluing (e.g. by epoxy glue) or by any other suitable method. In this embodiment, no dialysate leaks outside the measurement cell 1206, and the transparency of the measurement cell is preserved. The cannula can be of varying length according to the needs of the user, relating to age, thickness of the tissue where cannula is inserted, properties of analyte and dialysate, etc.

In some embodiments of the invention, an optical method is used to detect glucose concentration levels. The optical method used may be any of the optical methodologies described below, or any combination of them.

For example, the sensor may be based on an optical method using Near-Infrared (NIR) spectroscopy. In NIR measurements, a selected band of near-infrared light is passed through the sample and the glucose concentration level is obtained from a subsequent analysis of the resulting spectrum. NIR transmission and reflectance measurements of glucose are based on the fact that glucose-specific properties are embedded within the NIR spectra and can be extracted by using multivariate analysis methods (see, for example, Diab Tech Ther 2004; 6(5): 660-697, Anal. Chem. 2005, 77: 4587-4594).

In another embodiment, the sensor(s) of a sensing apparatus according to embodiments of the present invention may be based on an optical method using mid-IR spectroscopy. This method stems from absorbance spectra in the mid-IR range. This range contains absorbance fingerprints generated by the highly specific and distinctive fundamental vibrations of biologically important molecules such as glucose, proteins, and water. Two strong bands of glucose are found at 9.25 and 9.65 μm. A method based on these strong mid-IR absorbencies can be used to measure glucose concentration levels.

In yet another embodiment, the sensor(s) may be based on light scattering measured by localized reflectance (spatially resolved diffuse reflectance) or NIR frequency domain reflectance techniques. In localized reflectance, a narrow beam of light illuminates a restricted area on the surface of a body part, and reflected signals are measured at several distances from the illumination point. Both localized reflectance measurements and frequency domain measurements are based on changes in glucose concentration, which affects the refractive index mismatch between the ISF and tissue fibers. This technique could be applied on measuring glucose concentration inside the transparent measurement cell, rather than through tissue.

In another embodiment, the sensor(s) may be based on Raman spectroscopy for the detection of glucose, which measures the intrinsic property of the glucose molecule. The Raman effect is a fundamental process in which energy is exchanged between light and matter. In Raman spectroscopy the incident light, often referred to as ‘excitation’ light, excites the molecules into vibrational motion. Since light energy is proportional to frequency, the frequency change of this scattered light must equal the vibrational frequency of the scattering molecules. This process of energy exchange between scattering molecules and incident light is known as the Raman effect. The Raman scattered light can be collected by a spectrometer and displayed as a ‘spectrum’, in which its intensity is displayed as a function of its frequency change. Since each molecular species has its own unique set of molecular vibrations, the Raman spectrum of a particular species will consist of a series of peaks or ‘bands’, each shifted by one of the characteristic vibrational frequencies of that molecule. Thus, Raman spectroscopy can be employed to accurately measure tissue and blood concentrations of glucose (see, for example, Phys. Med. Biol. 2000 45 (2) R1-R59).

In another embodiment, glucose levels may be measured by a fluorescence energy transfer (FRET)-based assay for glucose, where concanavalin A is labeled with the highly NIR-fluorescent protein allophycocyanin as donor and dextran labelled with malachite green as the acceptor (see, J Photochem Photobiol 2000; 54: 26-34. and Anal Biochem 2001; 292: 216-221). Competitive displacement of the dextran from binding to the lectin occurs when there are increasing glucose concentrations, leading to a reduction in FRET, measured as intensity or lifetime (time-correlated single-photon counting).

In another embodiment, the sensor(s) may be based on a photoacoustic method. Photoacoustics (PA) involves ultrasonic waves created by the absorption of light. A medium is excited by a laser pulse at a wavelength that is absorbed by a particular molecular species in the medium. Light absorption and subsequent radiationless decay cause microscopic localized heating in the medium, which generates an ultrasound pressure wave that is detectable by a hydrophone or a piezoelectric device. Analysis of the acoustic signals can map the depth profile of the absorbance of light in the medium. Glucose trends can be tracked by the photoacoustic technique which can work as a noninvasive instrument for the monitoring of blood glucose concentrations (see Clin Chem 1999 45(9): 1587-95).

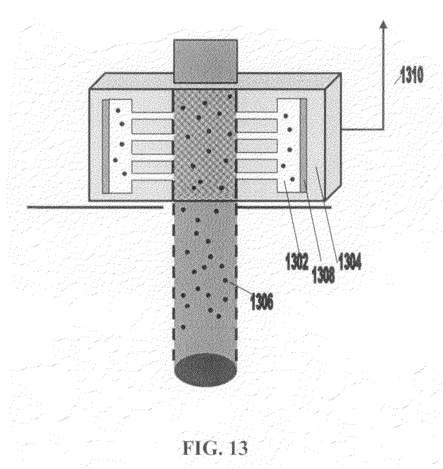

FIG. 13 illustrates an embodiment containing an electrochemical sensor 1302. In this embodiment, the sensing apparatus 1304 may be used to measure the concentration of glucose 1306 within the dialysate using a chemical reaction with glucose oxidase (GOX), producing an electrical current relative to the concentration of glucose in the interstitial fluid ISF. In this embodiment, the glucose sensor is coupled with an enzymatic membrane 1308, containing glucose oxidase (GOX). The reaction of the glucose-rich dialysate with the GOX eventually creates an electrical current flow, translated to a value corresponding to the glucose concentration level in the measured compartment (ISF, blood, etc.).

In another embodiment, the sensing apparatus may be based on use of a constituent mixed within the dispensing fluid at a predetermined concentration. The constituent has chemical or optical characteristics changed upon interaction with glucose, or any other measured molecule, where the end product of the reaction could be measured optically (using spectroscopic analysis) or chemically.

In another embodiment, the sensing apparatus may be based on any combination of several methods. This may include any combination of optical methods, non-optical methods and electrochemical methods. For example, such a combination could include of two optical methods, or an optical method with a non-optical method e.g. ultrasound-based method.

In any of the above-described embodiments, the sensing apparatus 1304 may be used to measure the concentration of glucose present in the dialysate to produce a signal indicating the detected glucose level. This output signal may be used as feedback 1310 to a processor-controller apparatus, which controls the operation of a dispensing apparatus.

The closed loop system embodiments may each include a single compact case which includes the dispensing apparatus, the fluid reservoir, tubing and pump, the sensing apparatus, the cannula and sensing device, and the processor-controller apparatus.

Thus it is seen that systems and methods are provided for sensing analyte and dispensing therapeutic fluid. Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The inventors reserve the right to pursue such inventions in later claims. Below are listed only some of the modifications and advantages, which are within the scope of the invention.

a) It is not necessary to wait for the establishment of complete concentration equilibrium—in the allowable time frame, a process (e.g., performed by computer program code stored in memory) can be used to approximate the partial equilibrium of analyte concentration to the complete equilibrium concentration.

b) A single cannula may be used as fluid delivery means and as sensing means.

c) The delivered drug (i.e. insulin) may function as the perfusate allowing diffusion of an analyte (i.e. glucose) within the body (i.e. ISF), and thus utilized as a measurement fluid.

d) The semi permeable cannula may allow osmotic differentiation between molecules of different sizes.

e) The optical measurement may be done in a completely transparent measurement cell without distortion of the signal by the surrounding tissue.

f) Flow of the dispensed drug, or fluid, may “wash” the cannula and prevent occlusion.

Any and all articles, patents, patent applications, and/or publications recited in the present application are all hereby incorporated by reference herein in their entireties.

What is claimed is:

1. Apparatus for in vivo detection of an analyte, comprising:
at least one housing;
a cannula comprising a proximal portion located within the housing and a distal portion located external to the housing, wherein the distal portion is configured for subcutaneous placement within a mammal's body and at least a portion of said cannula is permeable to molecules of an analyte;
a sensor configured to detect a concentration level of the analyte within the cannula; and
a pump residing in the housing and adapted to transport a fluid to the cannula.

2. The apparatus of claim 1, wherein the analyte comprises glucose.

3. The apparatus of claim 1, wherein the sensor is located at least partially within the housing and is configured to detect the concentration level of the analyte within a proximal portion of the cannula.

4. The apparatus of claim 3, wherein the pump is configured to transport a perfusate fluid.

5. The apparatus of claim 4, wherein said perfusate fluid is selected from the group consisting of a therapeutic fluid, a non-therapeutic fluid and a combination thereof.

6. The apparatus of claim 4, wherein said perfusate fluid comprises insulin.

7. The apparatus of claim 4, wherein said perfusate fluid comprises saline.

8. The apparatus of claim 1, wherein the sensor is configured to detect a concentration level of the analyte at about, or subsequent to, the establishing of a concentration equilibrium between the analyte within the cannula and the analyte outside the cannula.

9. The apparatus of claim 1, wherein the sensor comprises an optical sensor.

10. The apparatus of claim 9, wherein the optical sensor is configured to detect the concentration level of the analyte based on an optical detection method selected from the group of optical detection methods consisting of near infra red (“NIR”) reflectance, mid infra red (“IR”) spectroscopy, light scattering, Raman scattering, fluourescence measurements, and a combination thereof.

11. The apparatus of claim 1, wherein the sensor is selected from the group consisting of an optical sensor, electrochemical sensor, acoustic sensor and a combination thereof.

12. The apparatus of claim 1, further comprising a memory capable of storing at least concentration levels detected by the sensor continuously or at predetermined intervals.

13. The apparatus of claim 1, wherein the housing comprises a patch that is cutaneously adherable to the mammal's body.

14. The apparatus of claim 1, wherein the distal portion of the cannula is configured for subcutaneous placement within a location of the mammal's body that provides access to interstitial fluid (“ISF”).

15. The apparatus of claim 1, wherein the distal portion of the cannula is configured for subcutaneous placement within a location of the mammal's body that provides access to blood.

16. The apparatus of claim 4, wherein the housing further comprises:
a processor; and
a reservoir for the perfusate fluid,
wherein the pump is in fluid communication with the reservoir and in electrical communication with the processor, wherein the pump is configured to transport the perfusate fluid to the cannula in an amount based at least in part on a signal received from the processor.

17. The apparatus of claim 1, wherein the pump comprises a peristaltic pump.

18. Apparatus for in vivo detection of an analyte, comprising:
a cannula comprising a proximal portion located within a housing and a distal portion located external to the housing, wherein the distal portion is configured for subcutaneous placement within a mammal's body and at least a portion of said cannula is permeable to molecules of an analyte; and
a sensing means, which is configured to detect a concentration level of the analyte within the cannula.

19. Apparatus for in vivo detection of an analyte and delivery of a therapeutic fluid to the mammal's body, comprising:
a housing comprising at least a sensor, a pump, a processor and a reservoir for the therapeutic fluid; and
a cannula comprising a proximal portion located within the housing and a distal portion located external to the housing, wherein the distal portion is configured for subcutaneous placement within a mammal's body and at least a portion of said first cannula is permeable to molecules of an analyte;
wherein the sensor is in communication with the processor and is configured to detect a concentration level of the analyte within the proximal portion of the cannula; and
wherein the pump is in fluid communication with the reservoir and in electrical communication with the processor and is configured to deliver the therapeutic fluid to the mammal's body according to the detected concentration level.

20. The apparatus of claim 19, further comprising a second cannula which is in communication with the mammal's body, and wherein the pump is configured to deliver the therapeutic fluid to the mammal's body through the second cannula.

21. The apparatus of claim 19, wherein the sensor and the pump operate within a closed-loop configuration.

22. The apparatus of claim 19, wherein the sensor and the pump operate within a semi-closed loop configuration upon external input.

23. The apparatus of claim 19, wherein the housing comprises a patch that is cutaneously adherable to the mammal's body.

24. A method for in vivo detection of an analyte, comprising:
providing a cannula at least a portion of which is permeable to molecules of an analyte;
positioning the cannula at least partially subcutaneously within a mammal's body;
transporting a fluid to the cannula; and
sensing a concentration level of the analyte within the cannula at about, or subsequent to, establishing an equilibrium between a concentration level of the analyte within the cannula and a concentration level of the analyte outside the cannula.

25. The method of claim 24, wherein said fluid is a perfusate fluid.

26. The method of claim 25, wherein said perfusate fluid is selected from the group consisting of a therapeutic fluid, a non-therapeutic fluid and a combination thereof.

27. The method of claim 25, wherein said perfusate fluid comprises insulin.

28. The method of claim 25, wherein said perfusate fluid comprises saline.

29. The method of claim 25, wherein the analyte comprises glucose.

30. The method of claim 25, wherein the sensing of the concentration level is carried out within the proximal portion of the cannula and wherein the method further comprises transporting the analyte to the proximal portion of the cannula.

31. The method of claim 25, wherein the sensing is selected from the group consisting of optical sensing, electrochemical sensing, acoustical sensing, and a combination thereof.

32. A method for in vivo detection of an analyte and for delivery of a fluid to a mammal's body comprising
providing a cannula at least a portion of which is permeable to molecules of the analyte;
positioning the cannula at least partially subcutaneously within the mammal's body;
transporting the fluid to the cannula;
detecting a concentration level of the analyte within the cannula at about, or subsequent to, establishing an equilibrium between concentration level of the analyte within the cannula and concentration level of the analyte outside the cannula; and
delivering the fluid to the mammal's body in an amount based at least in part on the detected concentration level.

33. The method of claim 32, further comprising providing a second cannula, wherein the delivering of the fluid is carried out through the second cannula.

34. The method of claim 32, wherein said fluid is a perfusate fluid.

35. The method of claim 34, wherein said perfusate fluid is selected from the group consisting of a therapeutic fluid, a non-therapeutic fluid and a combination thereof.

36. The method of claim 35, wherein said therapeutic fluid comprises insulin.

37. The method of claim 34, wherein said perfusate fluid comprises saline.

38. The method of claim 32, wherein the analyte comprises glucose.

39. The method of claim 32, wherein the sensing of the concentration level of the analyte is carried out within the proximal portion of the cannula and wherein the method further comprises transporting the analyte to the proximal portion of the cannula.

40. The method of claim 32, wherein the sensing is selected from the group consisting of optical sensing, electrochemical sensing, acoustical sensing, and a combination thereof.
